(19) United States
(12) Patent Application Publication
(10) Pub. No.: US 2003/0095953 A1
(43) Pub. Date: May 22, 2003

(54) METHODS OF REVERSING DRUG RESISTANCE IN CANCER CELLS

(76) Inventors: MYLES C. CABOT, LOS ANGELES, CA (US); YONG-YU LIU, LOS ANGELES, CA (US)

Correspondence Address: CAROL M GRUPPI, MCCUTCHEN DOYLE BROWN & ENERSEN LLP, THREE EMBARCADERO CENTER, SAN FRANCISCO, CA, 941114066

(21) Appl. No.: 09/439,293

(22) Filed: Nov. 12, 1999

(51) Int. Cl. A61K 48/00

(52) U.S. Cl. 424/93.21; 514/44

(57) ABSTRACT

This invention relates in general to methods and compositions for reversing the drug resistance of cancer cells. In particular this invention is directed to inhibition of drug resistance in cancer cells or to the induction of apoptosis in cancer cells by the use of glucosylceramide synthase antisense compounds. This invention is further directed to compositions comprising glucosylceramide synthase antisense compounds and a kit or drug delivery system comprising the compositions.

### 1. FIELD OF THE INVENTION

The present invention relates to methods of reversing drug resistance in cancer cells. More specifically, this invention relates to a method of reversing drug resistance in cancer cells or the induction of apoptosis in cancer cells by the use of glucosylceramide synthase antisense compounds and compositions and kits comprising the same.

### 2. BACKGROUND OF THE INVENTION

More than two million new cases of cancer are reported annually in the seven major worldwide pharmaceutical marketplaces (US, Japan, Germany, Italy, France, Spain, UK) (Krul, (1994) Emerging Resources, Decision resources Inc.,, pp:79-94). Chemotherapy is an important part of modern clinical cancer treatment for human malignancies. However, chemotherapy frequently is ineffective due to either endogenous or acquired tumor cell resistance. Typically, the resistance is developed simultaneously to a wide range of structurally unrelated chemotherapeutic drugs with different mechanisms of action and therefore is called multidrug resistance (MDR) (Deuchards and Ling, (1989) Seminars in Oncology 316:1385-1393; Pastan and Gottesman, (1987) New Engl. J. of Med 16, 156-165). Generally, only 5-10% of new cancer cases will respond successfully to chemotherapy, and 40-45% of cancer patients will annually develop MDR to their particular chemotherapeutic regimens.

Several mechanisms can account for the multifactorial nature of MDR at the molecular and cellular level (Ling, V. (1992) Cancer 69:2603-2609). Decreased drug uptake or increased drug efflux, altered redox potential, enhanced DNA repair, increased drug sequestration mechanisms or amplification of the drug-target protein all are postulated cellular mechanisms for expression of cancer cell drug resistance to various chemotherapeutic agents. One of the most thoroughly studied mechanisms by which tumor cells acquire MDR is overexpression of a transmembrane glycoprotein, called P-glycoprotein (Pgp). Pgp is thought to act by rapidly pumping hydrophobic chemotherapeutic agents out of tumor cells, thereby decreasing intracellular accumulation of certain chemotherapeutic agents below their cytostatic concentrations. Thus, the approach most extensively employed in an attempt to circumvent multidrug resistance has involved the use of resistance modifers such as verapamil to reverse Pgp function. However, so far this approach has had limited clinical impact (Volm (1998) Anticancer Res 18:2905-2918).

Recently, it has been shown that MDR cells, as opposed to drug-sensitive cells, display increased levels of glucosylceramide (Lavie, Y et al., (1996) J. Biol. Chem 271:19530-19536271:19530-19536) and further that MDR modulators may increase the cellular susceptibility to chemotherapeutic agents through regulation of ceramide metabolism in cancer cells (Lavie, Y et al., (1997) J. Biol. Chem 272:1682-1687). Glucosylceramides are glycolipids that are produced by glucosylceramide synthase (GCS) transferring glucose from UDP-glucose to ceramide (Basu, et al., (1968) J. Biol. Chem 243:5802-5804). In addition to being the building blocks of biological membranes, glycosphingolipids appear to be involved in cell proliferation (Hannun and Bell, (1989) Science, 243:500-507) differentiation (Schwarz, A. et al., (1995) J. Biol. Chem. 270:10990-10998; Harel and Futerman, (1993) J. Biol. Chem. 268:14476-14481), oncogenic transformation (Hakomori, S. (1981) Annu. Rev. Biochem. 50: 733-764; Morton, D. L. et al., (1994) Prog. Brain Res. 101: 251-275) and the prevention of the onset of apoptosis (Nakamura, S. et al., (1996) J. Biol. Chem.. 271: 1255-1257).

Apoptosis or programmed cell death is widely recognized to be a cellular mechanism crucial for toxic response to chemotherapeutic agents (Wyllie, A. H. (1997) Eur. J. Cell Biol. 73:189-197). A substantial body of evidence now exists defining ceramide as a messenger for the induction of apoptosis. In intact cells, rapid ceramide generation is an early event in the apoptotic response to numerous stimuli including cytokines and environmental stresses, and ceramide analogs mimic the effect of stress and induce apoptosis (Hannun, Y. (1994) J. Biol. Chem. 269:3125-3128; Kolesnick and Golde, (1994) Cell 77:325-328; Hannun and Obeid, (1995) Trends in Biochem Sci. 20:73-77; Jarvis, W. D. et al., (1996) Clin. Cancer Res.2:1-6). Loss of ceramide production is one cause of cellular resistance to apoptosis induced by either ionizing radiation, or tumor necrosis factor-α and adriamycin (Hannun, Y. A. (1997) Blood 89, 1845-1853; Chuma, S. J.et al (1997) Cancer Res. 57: 1270-1275; Bose R., et al. (1995) Cell 82: 405-414; Cai, Z.et al., (1997) J. Biol. Chem. 272: 6918-6926; Santana P.et al (1996) Cell 86; 189-199; Liu, Y. Y.et al. (1999) J. Biol. Chem. 274: 1140-1146). Accumulation of glucosylceramide (GC), a simple glycosylated form of ceramide, is a characteristic of some MDR cancer cells and tumors derived from patients who are less responsive to chemotherapy (Lavie, Y.et al. (1996) J. Biol. Chem. 271: 19530-19536; Lucci A.et al. (1998) Anticarcer Res. 18: 475-480). Modification of ceramide metabolism, by blocking the glycosylation pathway, has been shown to increase cancer cell sensitivity to cytotoxics (Lucci, A.et al. (1999) Int. J. Onc. 15: 541-546; Lavie, Y., et al. (1997) J. Biol. Chem. 272: 1682-1687; Lucci, A., et al. (1999) Cancer 86: 299-310). Further, drug combinations that enhance ceramide generation and limit glycosylation have been shown to enhance kill in cancer cell models (Lavie, Y.et al., J Biol.. Chem. 272: 1682-1687; Lucci, A., et al. (1999) Cancer 86: 299-310). Other work has shown that ceramide toxicity can be potentiated in experimental metastasis of murine Lewis lung carcinoma and human neuroepithelioma cells by inclusion of a glucosylceramide synthase inhibitor (Inokuchi, J., et al. (1990) Cancer Res. 50: 6731-6737; Spinedi, A., et al. (1998) Cell Death Differ. 5: 785-791).

Antisense compounds, such as antisense oligonucleotides and antisense gene transfection have been shown to have a therapeutic effect with less adverse effects relative to conventional therapies. (Stein, C. A. and Cheng, Y. C. (1993) Science 261:1004-1012; Alama, A. et al (1997) Pharmacological Res. 89:171-178; Ziegler, A. et al (1997) J. Natl. Cancer Inst. 89:1027-36). Antisense nucleic acids have been employed to modulate the expression of oncogenes, such as c-myc (Yokoyama, K. and Imamoto, F. (1988) Proc. Natl. Acad. Sci. USA 83:7365), c-fos (Holt, J. T. et al (1986) Proc. Natl. Acad. Sci. USA 83:4794), C-myb (Gewirtz, A. M. and Calabretta, B. (1988) Science 242:1303), c-ras (Tidd, D. M. et al (1988) Anti-Cancer Drug Design 3:117), c-raf-1 (Kasid, U. et al (1989) Science 240:1354) and p53 (Shohat, O. et al (1987) Oncogene 1:277). Cancer Res. 54:2218-2222). Glioblastoma cells, which expressed an antisense RNA to the IGF-1 receptor, are non-tumorigenic and induce regression of wild-type tumor (Resnicoff, M. et al (1994) Cancer Research 54: 2211-2222) and antisense k-ras inhibits pancreatic tumor dissemination. (Aoki, K. et al (1995) Cancer Res. 55:3810-3816). Bcl-2 antisense has been shown to sensitize multidrug resistance in small-cell lung cancer cells (Ziegler, A. et al (1997) J. Natl. Cancer Inst. 89:1027-36), and in human melanoma in SCID mice (Jansen, B. et al (1998) Nature Medicine 4:232-234). As a result of the role that ceramide is believed to play in drug resistance there is a great desire to provide compounds capable of modulating ceramide metabolism and thus apoptosis. Antisense componds capable of modulating ceramide metabolism and thus apoptosis would have tremendous therapeutic utility for cancer and a wide variety of diseases, where regulation of apoptosis and proliferative capacity of are tightly coupled.

### 4. SUMMARY OF THE INVENTION

This invention relates in general to methods and compositions for reversing drug resistance of a cancer cell, thereby restoring chemotherapy sensitivity. More particularly this invention is directed to reversal of drug resistance in a cancer cell or to the induction of apoptosis in cancer cells by the use of glucosylceramide synthase antisense compounds. This invention is also directed to a method of reversing drug resistance in a cancer cell or inducing apoptosis in a cancer cell in a subject by administering glucosylceramide synthase antisense compounds. This invention is further directed to compositions and kits comprising glucosylceramide synthase antisense compounds.

It is an object of this invention to provide a method of reversing drug resistance in a cancer cell by introducing a glucosylceramide synthase antisense compound into the cancer cell.

It is another object of this invention to provide a method of reversing drug resistance in a cancer cell by introducing a glucosylceramide synthase antisense compound into the cancer cell and contacting the cancer cell with at least one other agent, or a combination thereof.

Another object of the invention is directed to a method of inducing apoptosis in a cancer cell, by introducing a glucosylceramide synthase antisense compoundinto a cancer cell.

Yet another object of the invention is directed to a method of inducing apoptosis in a cancer cell, by introducing a glucosylceramide synthase antisense compound into the cancer cell and contacting the cancer cell with and at least one other agent, or combination thereof.

Yet a further object of the invention is to provide a method of reversing drug resistance in a cancer cell or inducing apoptosis in a cancer cell in a subject by administering glucosylceramide synthase antisense compounds either alone or in conjunction with at least one other agent or combiation thereof.

It is another object of this invention to provide compositions for use in the methods described herein.

It is a further object of this invention to provide a kit or drug delivery system comprising the compositions for use in the methods described herein.

### 5. DESCRIPTION OF THE FIGURES

FIGS. 1A-1C show the structure of pcDNA 3.1/his A-asGCS (antisense glucosyl ceramide synthase), GCS enzyme activity, and cellular photomicrographs of parent and transfected variants. FIG. 1A shows the structure of pcDNA3.1/his A-asGCS. GCS antisense was inserted into the EcoR I site of pcDAN3.1/his A. Recombinant asGCS was fused with Xpress tag, and expression was driven by CMV promoter. FIG. 1B shows glucosylceramide synthase activity in parent and in MCF-7-AdrR (Adriamycin resistant cells)/asGCS cells. GCS was assayed as detailed in Experimental Procedures. AdrR, MCF-7-AdrR cells; Vector, MCF-7-AdrR cells transfected with pcDNA 3.1/his A (vector control); asGCS, MCF-7-AdrR/asGCS cells, MCF-7-AdrR cells transfected with pcDNA 3.1/his A-asGCS. *p<0.001, compared with MCF-7-AdrR cells. FIG. 1C shows photomicrographs of MCF-7-AdrR and MCF-7-AdrR/asGCS cells. Cells were stained with Giemsa reagent and photographed at 200× magnification.

FIGS. 2A-2B show the expression of GCS mRNA and protein in MCF-7-AdrR cell variants. FIG. 2A shows mRNA expression of GCS. Isolated mRNA (5 ng) was amplified by high fidelity RT-PCR. The reverse PCR product, a 300-bp fragment of GCS, was resolved on 1% agarose gel electrophoresis, and stained with ethidium bromide (top strip). Housekeeper gene, β-actin was used as a control for even loading (bottom strip). Control lane, RT-PCR product without cellular mRNA; MCF-7-AdrR, MCF-7-AdrR parental cells; AdrR/asGCS, MCF-7-AdrR GCS antisense transfected cells. FIG. 2B shows GCS Western blot. GCS (50 μg protein/lane) was resolved using 4-20% SDS-PAGE, and reacted with GCS polyclonal antibody (1:1,000). AdrR/GCS, MCF-7-AdrR cells transfected with GCS cDNA (pcDNA 3.1/his A-GCS); MCF-7-AdrR, the parent cell line; AdrR/asGCS, GCS antisense-transfected MCF-7-AdrR cells. FIG. 2C shows western blots of anti-Xpress antibody. Blots were done as described above. The Xpress fused protein was reacted with Xpress antibody (1:500). Abbreviations as in FIG. 2B.

FIGS. 3A-3B show adriamycin and ceramide toxicity in MCF-7-AdrR and in GCS antisense-transfected MCF-7-AdrR cells. FIG. 3A shows cytotoxicity of adriamycin. Cells were seeded into 96-well plates and treated the following day with adriamycin, at the concentrations shown, in 5% FBS RPMI-1640 medium. After 72 hr exposure, cell viability was determined. Data represent the mean±SD of six replicates from three independent experiments. *,p<0.0001 compared with MCF-7-AdrR cells. FIG. 3B shows cytotoxicity of adriamycin and C₆-ceramide in the MCF-7-AdrR variants. The same conditions cited above were employed, except that C₆-ceramide was used in place of adriamycin. *p<0.0001 compared with MCF-7-AdrR cells; Adr, adriamycin.

FIGS. 4A-4B show cellular ceramide metabolism under Adriamycin Stress. FIG. 4A shows influence of time in presence of adriamycin on cellular ceramide metabolism. Cells were seeded in 6-well plates in 5% FBS RPMI-1640 medium without or with adriamycin (2.5 μM) for the indicated times. [3H]Palmitic acid was added for the initial or final 24 hr period. FIG. 4B shows the effect of adriamycin dose on cellular ceramide metabolism. Cells were treated with increasing concentrations of adriamycin for 48 hr, and radiolabeled simultaneously during the last 24 hr period. *p<0.001, compared to MCF-7-AdrR cells.

FIG. 5 shows Caspase-3 Activity under Adriamycin stress. Cells were treated without or with adriamycin (10 μM) for 24 and 48 hr. After harvest, the soluble fraction obtained after cell lysis (10⁶ cell/tube) was incubated with DEVD-AFC substrate at 37° C. for 60 min as detailed in Experimental Procedures. The fluorescence of cleaved AFC was measured at 505 nm. *p<0.0001, compared to MCF-7-AdrR cells treated with adriamycin for each corresponding treatment period.

FIGS. 6A-6B show P-glycoprotein and Bcl-2 expression in MCF-7-AdrR and MCF-7-AdrR/as GCS cells. Detergent-soluble cellular protein was isolated from the respective cell lines and subjected to SDS-PAGE (50 μg/lane). Protein was transferred to nitrocellulose, and the immunoblot was incubated with the specified antibody. FIG. 6A shows P-glycoprotein Western blots. C219 monoclonal antibody was used to recognize P-glycoprotein. FIG. 6B shows Bcl-2 Western blots. Ab-1 monoclonal antibody was utilized to blot Bcl-2 protein. MCF-7 cells were used as a positive control for Bcl-2.

### 6. DETAILED DESCRIPTION OF THE INVENTION

6.1 Definitions

The term “nucleic acid” refers to, deoxyribonucleic acid (DNA), ribonucleic acid (RNA), and also cDNA.

The term “oligonucleotide” refers to an oligomer or polymer of RNA or DNA. The oligonucleotide may comprise naturally occurring nucleotides or modified nucleotides or combinations thereof. The oligonucleotide may be linear or circular, linear being the prefered formation.

The term “cDNA” refers to all nucleic acids that share the arrangement of sequence elements found in native mature mRNA species, where sequence elements are exons and 3′ and 5′ non-coding regions. Normally mRNA species have contiguous exons, with the intervening introns removed by nuclear RNA splicing, to create a continuos open reading frame encoding the protein.

The term “genomic sequence” refers to a sequence having non-contiguous open reading frames, where introns interrupt the protein coding regions. It may further include the 3′ and 5′ untranslated regions found in the mature mRNA. It may further include specific transcriptional and translational regulatory sequences, such as promoters, enhancers, etc., including about 1 kb, but possibly more, of flanking genomic DNA at either the 5′ or 3′ end of the transcribed region. The genomic DNA may be isolated as a fragment of about 100 kb or smaller, and substantially free of flanking chromosomal sequence.

The term “antisense compound” refers to a compound, preferably nucleic acids sequences, which modulate the expression of a gene. Generally, nucleic acid sequences complementary to the gene and the products of gene transcription are desiginated “antisense”, and nucleic acid sequences having the same sequence as the transcript or being produced as the transcript are designated “sense”. The antisense compound preferably modulates either gene or protein expression and impairs the function of the protein.

The term “modulation” refers to or means either an increase or a decrease in the expression of a gene transcript or protein or impairment of the activity of the protein.

The term “reverses drug resistance ” refers to a decrease, reduction, inhibition, prevention or abolition of drug resistance in cancer cells in vitro or in vivo or an enhanced sensitivity to a drug in cancer cells in vitro or in vivo. For example, an inhibition of drug resistance may be characterized by a reduction in the amount of chemotherapeutic drug used on the cancer cell, while achieving the same degree of effectiveness or be reestablishing sensitivity to a chemotherapeutic agent in cancer cells which had become refractory to that chemotherapeutic agent.

The term “specifically hybridizable” is used to indicate a sufficient degree of complementarity or precise pairing such that stable and specific binding occurs between the oligonucleotide and the DNA or RNA target. It is understood in the art that the sequence of an antisense compound need not be 100% complementary to that of its target nucleic acid to be specifically hybridizable. By way of example the antisense nucleic acid compound may be between about 60% to about 100% complementary or between about 70% to about 90% complementary.

The term “disease” refers to a variety of diseases such as, cancer, autoimmune diseases, or any condition characterized by inappropriate cellular proliferation, such as in diseases of the skin (e.g., psoriasis or hyperkeratosis). By way of example, a disease may involve a tumor. Generally, a tumor benign or malignant exhibits abnormal or excessive cellular proliferation. The tumor may be characterized as benign if it has not spread beyond its anatomical locus and cancerous or malignant if it has invaded the surroundings of its original anatomical locus and spread to other sites. Both benign and cancerous tumors are intended to be encompassed by this invention. Also intended to be included are, viral infection (e.g., HIV), bacterial infection or fungal infection.

The term “cancer” includes a myriad of diseases, including, but not limited to, breast cancer, melanoma, epithelial cell derived cancers, lung cancer, colon cancer, ovarian cancer, kidney cancer, prostate cancer, brain cancer, or sarcomas. Such cancers may be caused by, chromosomal abnormalities, degenerative growth and developmental disorders, mitogenic agents, ultraviolet radiation (UV), viral infections, inappropriate tissue expression of a gene, alterations in expression of a gene, or carcinogenic agents.

The term “subject” refers to any animal, preferably a mammal, preferably a human. Veterinary uses are also intended to be encompassed by this invention.

The term “GCS activity” refers to the biological activities or function of the naturally occurring GCS enzyme.

The term “construct” refers to a recombinant nucleic acid, generally recombinant DNA, that has been generated for the purpose of the expression of a specific nucleotide sequence(s), or is to be used in the construction of other recombinant nucleotide sequences.

The term “operably linked” refers to a DNA sequence and a regulatory sequence(s) that are connected in such a way as to permit gene expression when the appropriate molecules (e.g., transcriptional activator proteins) are bound to the regulatory sequence(s).

The term operatively “operatively inserted” refers to a nucleotide sequence of interest that is positioned adjacent to a nucleotide sequence that directs transcription and translation of the introduced nucleotide sequence of interest.

The term “corresponds to” refers to homologous to or substantially equivalent to or functionally equivalent to the designated sequence.

The term “transformation” refers to a permanent or transient genetic change, preferably a permanent genetic change wherein exogenous genetic material is operably inserted and expressed, induced in a cell following incorporation of new DNA (i.e., DNA exogenous to the cell). Where the cell is a mammalian cell (preferably a rodent cell), a permanent genetic change is generally achieved by operative introduction of the DNA into the genome of the cell.

6.2 Methods of Reversing Drug Resistance or Inducing Apoptosis

The present invention provides a method for reversing drug resistance in a cancer cell or induction of apoptosis in a cancer cell by the use of one or more glucosylceramide synthase antisense compounds, as well as compositions and kits for use in such methods. These methods and compositions are based on an observation by the inventors that antisense nucleic acid sequences targeting glucosylceramide synthase are highly effective in reversing drug resistance in cancerous cells as well as inducing apoptosis in cancer cells.

Accordingly, one aspect of this invention relates to a method of reversing drug resistance in a cancer cell by modulating the activity of glucosylceramide synthase. Preferably the modulation causes a decrease in the activity of glucosylceramide synthase. The method of reversing drug resistance in a cancer cell comprises introducing an antisense glucosylceramide synthase compound into the cancer cell. Another aspect of this invention relates to a method of inducing apoptosis in a cancer cell by modulating the activity of glucosylceramide synthase. Preferably, as with the method of reversing drug resistance in a cancer cell, the modulation causes a decrease in the activity of glucosylceramide synthase. The method of inducing apoptosis in a cancer cell also comprises introducing an antisense glucosylceramide synthase compound into a cancer cell and contacting the cancer cell with at least one other agent. Examples of such agents include, but are not limited to, adriamycin, Vinca alkaloids, or taxanes. In both methods the desired endpoint of contact with the glucosylceramide antisense compound is an impairment or disruption of the activity of glucosylceramide synthase.

The glucosylceramide synthase antisense compound suitable for use in the disclosed methods may be any type of molecule capable of impairing the activity of glucosylceramide synthase. In a preferred embodiment, a nucleic acid sequence is used as the glucosylceramide antisense compound. Preferably the nucleic acid sequence comprising the glucosylceramide antisense compound is complementary to and selectively hybridizes to the sense strand of the glucosylceramide synthase gene or RNA (e.g., pre mRNA, mRNA etc) produced by transcription of the glucosylceramide synthase gene. Catalytic RNA molecules including, but not limited to, ribozymes, may also be used to target the mRNA of glycosylceramide synthase. Homologous recombination may also be utilized to impair the function of glycosylceramide synthase.

The antisense nucleic acid sequence used in the methods of the subject application may be of varying lengths. For example, the antisense nucleic acid sequence may be complimentary to the full length cDNA of glucosylceramide synthase. (see for e.g., Examples 1-3). Alternatively, the nucleic acid sequence may be an oligonucleotide complementary to portions of the coding or regulatory regions of glucosylceramide synthase. By way of example, the oligonucleotide may range in length from between about 12 nucleotides to about 25 nucleotides, or between about 15 to about 20 nucleotides. The antisense oligonucleotides contacted with the cancer cells may all be of the same sequence. Alternatively, the antisense oligonucleotides may be a variety of sequences complimentary to different coding or regulatory regions of the glucosylceramide synthase gene. By way of example, the antisense oligonucleotides may be constructed so the oligonucleotides compliment the coding region in an overlapping fashion.

The antisense nucleic acid sequences may be generated by conventional methodology including, but not limited to, chemical synthesis, enzyme digestion, PCR amplification, etc. The antisense nucleic acid sequence may be constructed based on the coding sequence or parts thereof of a wild-type glucosylceramide synthase gene, naturally occurring polymorphisms, or genetically manipulated sequences (i.e., deletions, substitutions or insertions in the coding or non-coding regions) or sequences encoding a truncated or altered glucosylceramide synthase gene. Examples of sequences for glucosylceramide synthase include, but are not limited to, the human cDNA for glucosylceramide synthase (Ichikawa, et al (1996) Proc. Natl. Acad. Sci. USA 93:4638-4643) or the mouse glucosylceramide synthase sequence (Ichikawa et al (1998) Biochem. Molec. Biol. Int. 44:1193-1202).

Modified antisense nucleic acid sequences may also be utilized in the methods of the subject application. Preferably the modified antisense nucleic acid sequences are the functional equivalent of the nonmodifid antisense sequences. The antisense nucleic acid sequence may be modified at any point in the sequence, for example, all along the length of the nucleic acid sequence and/or in the 5′ position and/or in the 3′ position. Preferred modifications include, but are not limited to, modifications which facilitate entry of the nucleic acid sequence into the cancer cell or modifications which protect the nucleic acid sequence from the cellular environment. Examples of such modifications include, but are not limited to, replacement of the phosphodiester bond with a phosphorothioate, phosphorodithioate, methyl phosphonate, phosphoramidate, phosphoethyl triester, butyl amidate, piperazidate, or morpholidate linkage to enhance the resistance of the nucleic acid sequence to nucleases, replacement of the phosphate bonds between the nucleotides with an amide bonds (e.g., peptide nucleic acids which are nucleobases that are attached to a pseudopeptide backbone), incorporation of non-naturally occurring bases partially or along the whole length of the nucleic acid sequence (e.g., U.S. Pat. Nos. 5,192,236; 5,977,343; 5,948,901; 5,977,341; herein incorporated by reference.) to enhance resistance to nucleases or improve intracellular absorption, or incorporation of hydrophobic substitutes such as cholesterol or aromatic rings, or polymers to the nucleic acid sequences to facilitate passage through the cellular membrane (e.g., U.S. Pat. Nos. 5,192,236; 5,977,343; 5,948,901; 5,977,341; herein incorporated by reference.). The antisense nucleic acid sequences may be modified utilizing materials and methods known to those in the art, Prefferred modifications include, but are not limited to, antisense phospherothioate oligodeoxy-nucleotide of GCS, and peptide nucleic acid of GCS antisense.

It is understood by one skilled in the art that the antisense nucleic acid sequences impair the activity of a gene in a variety of ways and via interaction with a number of cellular products. Examples include, but are not limited to, the hydrolysis action catalyzed by RNAse H, the formation of triple helix structures, interaction with the intron-exon junctions of pre-messenger RNA, hybridization with messenger RNA in the cytoplasm resulting in an RNA-DNA complex which is degraded by the RNAas H enzyme, or by blocking the formation of the ribosome-mRNA complex and thus blocking the translation, or antisense peptides or proteins produced from the sequence of GCS antisense, inhibit GCS function or regulate its activity.

It is yet another aspect of the invention to provide a method of reversing drug resistance in a cancer cell or inducing apoptosis in a cancer cell by introducing the cancer cell with a glucosylceramide synthase antisense compound and at least one other agent. The agent may be any type of molecule from, for example, chemical, nutritional or biological sources (e.g., extracts from plant or animal sources or extracts thereof). The agent may be naturally occurring or synthetically produced and may encompass numerous chemical classes, though typically they are organic molecules, preferably small organic compounds having a molecular weight of more than 50 and less than about 2,500 Daltons. Such molecules may comprise functional groups necessary for structural interaction with proteins or nucleic acids. By way of example, chemical agents may be novel, untested chemicals, agonists, antagonists, or modifications of known therapeutic agents.

The agents to be used in the methods of the subject application may also be found among biomolecules including, but not limited to, peptides, saccharides, fatty acids, antibodies, steroids, purines, pryimidines, toxins conjugated cytokines, derivatives or structural analogs thereof or a molecule manufactured to mimic the effect of a biological response modifier. The agents may be obtained from a wide variety of sources including libraries of synthetic or natural compounds. Alternatively, libraries of natural compounds in the form of bacterial, fungal, plant, and animal extracts are available or readily produced, natural or synthetically produced libraries or compounds are readily modified through conventional chemical, physical and biochemical means, and may be used to produce combinatorial libraries. Known pharmacological agents may be subjected to random or directed chemical modifications, such as acylation, alkylation, esterification, amidification, etc. to produce structural analogs. Chemotherapeutic agents and chemosensitizers are preferred agents. The agent may be contacted with the cancer cell either concurrently with the glucosylceramide synthase antisense compound or sequentially (i.e., before or after the glucosylceramide synthase antisense compound).

The methods described herein, may also be utilized to reverse drug resistance or induce apoptosis in any disease characterized by inappropriate cellular proliferation. In addition to cancer, examples include, but are not limited to, autoimmune diseases, or diseases of the skin (e.g., psoriasis or hyperkeratosis). Also intended to be included are, viral infection (e.g., HIV), bacterial infection or fungal infection. These methods may also be utilized in a variety of applications including, but not limited to, study of lipid matabolism in a cell.

6.3 Cells

A variety of cells may be used in the methods of the subject application. Preferably, the cells to be used in the disclosed methods exhibit inappropriate cellular proliferation, such as, cancer cells. Nonlimiting examples of cancer cells that may be used include, but are not limited to, breast, prostate, ovarian, sarcomas, lymphoma, melanoma, sarcoma, leukemia, retinoblastoma, hepatoma, myeloma, glioma, mesothelioma or carcinoma cells. The cancer cell may be contacted with one or more glucosylceramide synthase antisense compound either in vitro or in vivo.

By way of example, the cells used in the methods may be primary cultures (e.g., developed from biopsy or necropsy specimens) or cultured cell lines. If cultured cell lines are used, preferably the cell lines are mammalian cancer cells, most preferably human cancer cells. Examples of cell lines that may be used include, but are not limited to MCF-7 (a breast cancer cell line), MCF-7 AdrR (adriamycin resistant), OVCAR-3 (human ovarian cancer cell line), melanoma cell lines (e.g., M-10, M-24, M-101; John Wayne Cancer Institute, Santa Monica, Calif., U.S.A.) and MCF-7/GCS. Desirable cell lines are often commercially available (e.g. American Type Culture Collection, 10801 University Blvd., Manassas Va., 20110-2209), available from the National Cancer Institute (Rockville, Md., U.S.A.) or readily made by conventional technology. By way of example, MDR cell lines or cells exhibiting resistance to chemotherapy may be produced by continous exposure of cells to chemotherapeutic agents followed by cloning of the resistant cells or developed by gene transfection.

6.4 Vectors

Vectors suitable for use in expressing the antisense glucosylceramide synthase compound comprise at least one expression control element operably linked to the nucleic acid sequence encoding GCS. Expression control elements are inserted in the vector to control and regulate the expression of the nucleic acid sequence. Examples of expression control elements include, but are not limited to, lac system, operator and promoter regions of phase lamda, yeast promoters, and promoters derived from polyoma, adenovirus, retroviruses, or SV40. It will be understood by one skilled in the art the correct combination of required or preferred expression control elements will depend on the cells to be used.

The antisense nucleic acid sequence may be introduced into an appropriate vector for extrachromosomal maintenance or for integration into the host. Preferably a vector that allows for stable integration into the genome is used. Examples of such vectors, but are not limited to retroviral vectors, vaccinia virus vectors, adenovirus vectors, herpes virus vector, fowl pox virus vector, plasmids, YACs, or Tet on gene expression vector from Clontech (Palo Alto, Calif.).

The vector may further comprise additional operational elements including, but not limited to, leader sequences, termination codons, polyadenylation signals, and any other sequences necessary or preferred for the appropriate transcription and/or translation of the nucleic acid sequence encoding GCS.

It will be further understood by one skilled in the art that such vectors are constructed using conventional methodology (See e.g. Sambrook et al., (eds.) (1989) “Molecular Cloning, A laboratory Manual” Cold Spring Harbor Press, Plainview, N.Y.; Ausubel et al., (eds.) (1987) “Current Protocols in Molecular Biology” John Wiley and Sons, New York, N.Y.) or are commercially available.

The means by which the cells may be transformed with the construct comprising the antisense nucleic acid sequences include, but are not limited to, microinjection, electroporation, transduction or transfection with lipofection and calcium phosphate, particle bombardment mediated gene transfer, or direct injection of nucleic acid sequences or other procedures known to one skilled in the art (Sambrook et al. (1989) in “Molecular Cloning. A Laboratory Manual”, Cold Spring Harbor Press, Plainview, N.Y.). For various techniques for transforming mammalian cells, see Keown et al. 1990 Methods in Enzymology 185:527-537.

One of skill in the art will appreciate that vectors may not be necessary for the antisense oligonucleotides applications of the subject invention. Antisense oligonucleotides may be introduced into a cell, preferably a cancer cell, by a variety of methods, including, but not limited to, liposomes or lipofection (Thierry, A. R. et al (1993) Biochem Biophys Res Commun 190:952-960; Steward, A. J. et al (1996) Biochem Pharm 51:461-469) and calcium phosphate.

6.5 Diseases

The methods of the subject invention also relate to methods of reversing drug resistance in a cancer cell or inducing apoptosis in a cancer cell in a subject by administering glucosylceramide synthase antisense compounds either alone or in conjunction with another agent. While these methods are exemplified in cancer cells, the methods may be utilized in any disease characterized by inappropriate cell proliferation. These diseases include, but are not limited to, AIDS, AIDS related complex, Karposi sarcoma, leukemia, myelopathy, respiratory disorder such as asthma, autoimmune diseases such as systemic lupus erythematosus, and collagen diseases such as rheumatoid arthritis lipid metabolism disorders such as Gaucher disease. In preferred embodiments, the disease is a cancer, as for example, a lymphoma, melanoma, sarcoma, leukemia, retinoblastoma, hepatoma, myeloma, glioma, mesothelioma or carcinoma.

6.5 Pharmaceutical Compositions and Routes of Administration

Aqueous compositions of the present invention are comprised of an effective amount of the glucosylceramide synthase antisense compounds, either alone or in combination with another agent (for example, but not limited to a chemotherapeutic agent alone or in combination with a chemosensitizer.) Such compositions will generally be dissolved or dispersed in a pharmaceutically acceptable carrier or aqueous medium. The terms “pharmaceutically or pharmacologically acceptable” refer to molecular entities and compositions that do not produce an adverse, allergic or other untoward reaction when administered to an animal, or human, as appropriate. As used herein, “pharmaceutically acceptable carrier” includes any and all solvents, dispersion media, coatings, antibacterial and antifungal agents, isotonic and absorption delaying, agents and the like. The use of such media and agents for pharmaceutical active substances is well known in the art. Except insofar as any conventional media or agent is incompatible with the active ingredients, its use in the therapeutic compositions is contemplated. Supplementary active ingredients, such as other chemotherapeutic agents, classical MDR modulators, and newer MDR modulators can also be incorporated into the compositions.

The active compounds of the present invention can be formulated for parenteral administration, e.g., for injection via the intravenous, intramuscular, sub-cutaneous, intratumoral or intraperitoneal routes. The preparation of an aqueous composition that contains a chemotherapeutic agent alone or in combination with a chemosensitizer as active ingredients will be known to those of skill in the art in light of the present disclosure. Typically, such compositions can be prepared as injectables, such as liquid solutions or suspensions. Solid forms, that can be formulated into solutions or suspensions upon the addition of a liquid prior to injection, as well as emulsions, can also be prepared.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof, as well as in oils. Under ordinary conditions of storage and use, these preparations contain a preservative to prevent the growth of microorganisms.

The pharmaceutical forms suitable for injectable use include (i) sterile aqueous solutions or dispersions, (ii) formulations including sesame oil, peanut oil or aqueous propylene glycol, and (iii) sterile powders for the extemporaneous preparation of sterile injectable solutions or dispersions. In all cases the form must be sterile and must be fluid to allow for easy use with a syringe. It must be stable under the conditions of manufacture and storage, and must be preserved against the contaminating action of microorganisms, such as bacteria and fungi.

The active compounds may be formulated into a composition in a neutral or salt form. Pharmaceutically acceptable salts include inorganic acids, e.g. hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents. Prolonged absorption of the injectable compositions can be brought about by including in the compositions agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In certain cases, the formulations of the invention could also be prepared in forms suitable for topical administration, such as in creams and lotions. These forms may be used for treating skin-associated diseases, such as various sarcomas.

Upon formulation, solutions will be administered in a manner compatible with the dosage formulation and in a therapeutically effective amount. The formulations are easily administered in a variety of dosage forms, such as the type of injectable solutions described above, with even drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject. Veterinary uses are also intended to be encompassed by this invention.

The antisense compounds, preferably antisense nucleic sequences, formulated by the methods described herein may be delivered to the target cancer cells or any cells characterized by inappropriate cellular proliferation by a variety of methods. Examples include, but are not limited to, introducing the antisense nucleic acid of the present invention into expression vector such as a plasmid or viral expression vector. Such constructs may be introduced into a cell, preferably a cancer cell, by calcium phosphate transfection, liposome (for example, LIPOFECTIN)-mediated transfection, DEAE Dextran-mediated transfection, polybrene-mediated transfection, or electroporation. A viral expression construct may be introduced into a cell, preferably a cancer cell, in an expressible form by infection or transduction. Such viral vectors include, but are not limited to, retroviruses, adenoviruses, herpes viruses and avipox viruses. Likewise, antisense oloigonucleotides may be introduced into cancer cells by a variety of methods. Examples include, but are not limited to, endoscopy, gene gun, or lipofection (Mannino, R. J. et al., 1988, Biotechniques, 6:682-690) Newton, A. C. and Huestis, W. H., Biochemistry, 1988, 27:4655-4659; Tanswell, A. K. et al., 1990, Biochmica et Biophysica Acta, 1044:269-274; and Ceccoll, J. et al. Journal of Investigative Dermatology, 1989, 93:190-194),.

An effective concentration of such antisense constructs or oligonucleotides may be administered topically, intraocularly, parenterally, orally, intranasally, intravenously, intramuscularly, subcutaneously or by any other effective means. In addition, the construct or oligonucleotide may be directly injected in effective amounts by a needle.

By way of example, antisense nucleic acid sequences, such as antisense constructs or antisense oligonucleotides may be contacted with cancer cells in a body cavity such as, but not limited to, the gastrointestinal tract, the urinary tract, the pulmonary system or the bronchial system via direct injection with a needle or via a catheter or other delivery tube placed into the cancer cells. Any effective imaging device such as X-ray, sonogram, or fiberoptic visualization system may be used to locate the target cancer cells tissue and guide the needle or catheter tube.

Alternatively, the antisense nucleic acids may be administered systemically (e.g., blood circulation, lymph system) to target cancer cells which may not be directly reached or anatomically isolated.

6.5 Kits

It is another object of this invention to provide compositions for use in the methods described herein. It is a further object of this invention to provide a kit or drug delivery system comprising the compositions for use in the methods described herein. All the essential materials and reagents required for reversing drug resistance or for inducing apoptosis in cancer cells, or for reversing inappropriate cellular proliferation, such as cancer cell proliferation, may be assembled in a kit. When the components of the kit are provided in one or more liquid solutions, the liquid solution preferably is an aqueous solution, with a sterile, aqueous solution being preferable

For in vitro or in vivo use, a glucosylceramide synthase antisense compound either alone or in combination with one or more agents such as a chemotherapeutic or chemosensitizer may be formulated into single or separate pharmaceutically acceptable compositions. In this case, the container means may itself be an inhalant, syringe, pipette, eye dropper, or other like apparatus, from which the formulation may be applied to an infected area of the body, such as the lungs, injected into an animal, or even applied to and mixed with the other components of the kit.

The components of these kits may also be provided in dried or lyophilized forms. When reagents or components are provided as a dried form, reconstitution generally is by the addition of a suitable solvent, which may also be provided in another container means. The kits of the invention may also include an instruction sheet defining administration of the chemotherapeutic agent and/or chemosensitizer to modulate glycolipid metabolism. or explaining the assays for determining sphingolipid levels in samples. The kits of the present invention also will typically include a means for containing the vials in close confinement for commercial sale such as, e.g., injection or blow-molded plastic containers into which the desired vials are retained. The kit may also contain instructions regarding use and administration of the antisense compounds comprising the kit. Irrespective of the number or type of containers, the kits may also comprise, or be packaged with, an instrument for assisting with the injection/administration or placement of the ultimate complex composition within the body of an animal. Such an instrument may be an inhalant, syringe, pipette, forceps, measured spoon, eye dropper or any such medically approved delivery vehicle. Other instrumentation includes devices that permit the reading or monitoring of reactions in vitro or in vivo.

All books, articles, or patents referenced herein are incorporated by reference. The following examples illustrate various aspects of the invention, but in no way are intended to limit the scope thereof.

### 7. EXAMPLES

7.1 Experimental Procedures

Materials. [3H]UDP-glucose (40 Ci/mmol) was purchased from American Radiolabeled Chemicals (St. Louis, Mo.). C6-Ceramide (N-hexanoylsphingosine) was purchased from LC Laboratories (Woburn, Mass.). Sulfatides (ceramide galactoside 3-sulfate) were from Matreya (Pleasant Gap, Pa.), and phosphatidylcholine (1,2-dioleoyl-sn-glycero-3-phosphocholine) was from Avanti Polar Lipids (Alabaster, Ala.). Adriamycin (doxorubicin hydrochloride), and other chemicals were purchased from Sigma (St. Louis, Mo.). FBS was purchased from HyClone (Logan, Utah). RPMI medium 1640 and DMEM medium (high glucose) were from Gibco BRL (Gaithersburg, Md.), and cultureware was from Corning Costar (Cambridge, Mass.). GCS antiserum (from rabbit) was kindly provided by Drs. D. L. Marks and R. E. Pagano (Mayo Clinic and Foundation, Rochester, Minn.). Anti-Xpress tag antibody was from Invitrogen (Carlsbad, Calif.). C219, the monoclonal antibody against P-glycoprotein, was from Signet Laboratories (Dedham, Mass.), and Bcl-2 monoclonal antibody (Ab-1) against human Bcl-2 was from Oncogene Research Products (Cambridge, Mass.).

Cell Lines and Culture Conditions. The human breast adenocarcinoma cell line, MCF-7-AdrR which is resistant to adriamycin (Cowan, K. H.et al.,. (1986) Proc. Natl Acad. Sci. USA, 83, 9328-32), was kindly provided by Dr. Kenneth Cowan and Dr. Merrill Goldsmith (National Cancer Institute, Bethesda, Md.). Cells were maintained in RPMI-1640 medium containing 10% (v/v) FBS, 100 units/ml penicillin, 100 μg/ml streptomycin, and 584 mg/liter L-glutamine. Cells were cultured in a humidified, 5% CO2 atmosphere tissue culture incubator, and subcultured weekly using trypsin-EDTA (0.05%-0.53 mM) solution. The stably transfected cells, MCF-7-AdrR/asGCS, were cultured in RPMI-1640 medium containing 400 μg/ml G418 (geneticin) in addition to the above components.

Giemsa staining. Giemsa staining was performed as described (Freshney, R. I (1994) Culture of Animal Cells: A Manual of Basic Technique. 3rd Ed. Wiley-Liss, Inc. New York, N.Y.). Cells were seeded in 60 mm dishes (105 cells/dish) in 10% FBS RPMI-1640 medium, and grown for 2 days at 37° C. After rinsing with PBS, cells were fixed with 50% methanol/PBS, followed by methanol, and stained with KaryoMAX Giemsa stain stock solution (Gibco BRL). Following washing with deionized water, cells were photomicrographed. The population doubling time of each cell line was measured. Briefly, cells were seeded in 24-well plates (104 cells/well) in 10% FBS RPMI-1640 medium and grown for 24, 48, 72 and 96 hr periods. After rinsing with PBS, cells were dispersed with trypsin/EDTA, suspended in medium and counted by hemocytometer.

pcDNA 3.1/his A-asGCS Expression Vector Construction and Transfection. pCG-2, a Bluescript II KS containing GlcT-1 (Ichikawa, S., et al., Proc. Natl. Acad. Sci. U.S.A. 93, 4638-4643 terminology for GCS) in the EcoR I site, was kindly provided by Dr. Shinichi Ichikawa and Dr. Yoshio Hirabayashi (The Institute of Chemical and Physical Research, Saitama, Japan). The full-length cDNA of human GCS was subcloned into the EcoR I site in the pcDNA 3.1/His A with Xpress™ tag peptide in the upstream region. Xpress tag fuses at the N-terminus of the cloned gene; therefore, GCS will be expressed as Xpress-GCS. Antisense orientation of GCS cDNA was analyzed with Vector NTI 4.0, and doubly checked by restriction digestion. When MCF-7-AdrR cells reached 20% confluence, pcDNA 3.1-asGCS (10 μg/ml, 100-mm dish) was introduced by co-precipitation with calcium phosphate (Mammalian Transfection Kit, Stratagene, La Jolla, Calif.). The transfected cells were selected in RPMI-1640 medium containing 10% FBS and 400 μg/ml G418. Each G418-resistant clone, isolated utilizing cloning cylinders, was propagated and later screened by GCS enzyme assay. pcDNA 3.1/his A plasmid, without GCS DNA, was used in control transfection.

Glucosylceramide Synthase Assay. To determine the levels of GCS in the G418-resistant clones, a modified radioenzymatic assay was utilized (Liu, Y. Y., et al.,. (1999) J. Biol. Chem. 274, 1140-1146, Shukla, G. S. and Radin N. S. (1990) Arch. Biochem. Biophys. 283, 372-378). Cells were homogenized by sonication in lysis buffer (50 mM Tris-HCl, pH 7.4, 1.0 μg/ml leupeptin, 10 μg/ml aprotinin, 25 μM PMSF). Microsomes were isolated by centrifugation (129,000×g, 60 min). The enzyme assay, containing 50 μg microsomal protein, in a final volume of 0.2 ml, was performed in a shaking water bath at 37° C. for 60 min. The reaction contained liposomal substrate composed of C6-ceramide (1.0 mM), phosphatidylcholine (3.6 mM), and brain sulfatides (0.9 mM). Other reaction components included sodium phosphate buffer (0.1 M) pH 7.8, EDTA (2.0 mM), MgCl2 (10 mM), dithiothreitol (1.0 mM), β-NAD (2.0 mM), and [3H]UDP-glucose (0.5 mM). Radiolabeled and unlabeled UDP-glucose were diluted to achieve the desired radiospecific activity (4,700 dpm/nmol). To terminate the reaction, tubes were placed on ice and 0.5 ml isopropanol and 0.4 ml Na2SO4 were added. After brief vortex mixing, 3 ml t-butyl methyl ether was added, and the tubes were mixed for 30 sec. After centrifugation, 0.5 ml upper phase which contained GC, was withdrawn and mixed with 4.5 ml EcoLume for analysis of radioactivity by liquid scintillation spectroscopy.

RNA Analysis. Cellular mRNA was purified using a mRNA isolation kit (Boehringer Mannheim, Indianapolis, Ind.). Equal amounts of mRNA (5.0 ng) were used for RT-PCR. Under upstream primer (5′-CCTTTCCTCTCCCCACCTTCCTCT-3′) and downstream primer conditions (5′-GGTTTCAGAAGAGAGACACCTGGG-3′), a 302 bp fragment in the 5′-terminal region of the GCS gene was produced using the ProSTAR HF single-tube RT-PCR system (High Fidelity, Stratagene) in a thermocycler (Mastercycler Gradient, Eppendorf). mRNA's were reverse transcribed using MMLV-reverse transcriptase at 42° C. for 15 min. DNA was amplified with TaqPlus Precision DNA polymerase in a 40 cycle PCR reaction, using the following conditions: denaturation at 95° C. for 30 sec; annealing at 60° C. for 30 sec and elongation at 68° C. for 120 sec. RT-PCR products were analyzed by 1% agarose gel electrophoresis stained with ethidium bromide. β-Actin (Gibco BRL) was used as control for even loading.

Cytotoxicity Assay. Assays were performed as previously described (Liu, Y. Y., et al., (1999) J. Biol. Chem. 274, 1140-1146; Lavie, Y. et al., (1997) J. Biol. Chem. 272, 1682-1687). Briefly, cells were seeded in 96-well plates (2,000 cells/well), in 0.1 ml RPMI-1640 medium containing 10% FBS, and cultured at 37° C. for 24 hr before addition of drug. Drugs were added in FBS-free medium (0.1 ml), and cells were cultured at 37° C. for the indicated periods. Drug cytotoxicity was determined using the Promega 96 Aqueous cell proliferation assay kit (Promega, Madison, Wis.). Absorbance at 490 nm was recorded using a Microplate Fluorescent Reader, model FL600 (Bio-Tek, Winooski, Vt.).

Analysis of Ceramide. Analysis was performed as previously described (Liu, Y. Y.et al., (1999) J. Biol. Chem. 274, 1140-1146, Lavie, Y., et al., (1996) J. Biol. Chem. 271, 19530-19536). Cells were seeded in 6-well plates (60,000 cells/well) in 10% FBS RPMI-1640 medium. After 24 hr, cells were shifted to 5% FBS medium with or without adriamycin, and grown for the indicated times. Cellular lipids were radiolabeled by adding [3H]palmitic acid (2.5 μCi/ml culture medium) for 24 hr. After removal of medium, cells were rinsed twice with PBS (pH 7.4), and total lipids were extracted as described (Lavie, Y. et al.,. (1996) J. Biol. Chem. 271, 19530-19536). The resulting organic lower phase was withdrawn and evaporated under a stream of nitrogen. Lipids were resuspended in 100 μl of chloroform/methanol (1:1, v/v), and aliquots were applied to TLC plates. Ceramide was resolved using a solvent system containing chloroform/acetic acid (90:10, v/v). Commercial lipid standards were co-chromatographed. After development, lipids were visualized by iodine vapor staining, and the ceramide area was scraped into 0.5 ml water. EcoLume counting fluid (4.5 ml) was added, the samples were mixed, and radioactivity was quantitated by liquid scintillation spectrometry.

Caspase-3 Assay. Caspase-3 activity was assayed by DEVD-AFC cleavage, using the ApoAlert Caspase-3 assay kit (Clontech, Palo Alto, Calif.). The assay was performed as previously described (Liu, Y. Y., et al., (1999) Exp. Cell Res. 252, 464-470). Cells were seeded in 100-mm dishes (500,000 cells/dish) in 10% FBS RPMI-1640 medium. After 24 hr, cells were shifted to 5% FBS RPMI-1640 medium without or with adriamycin, and grown for 24 and 48 hr. Following harvest, cells (106/vial) were lysed on ice for 10 min with 50 μl of lysis buffer, and cell debris was removed by centrifugation at 4° C., 10,000×g, for 5 min. The soluble fraction was incubated with 50 μM conjugated substrate DEVD-AFC in a 100 μl reaction volume at 37° C., for 60 min. The free AFC fluoresce was measured at λexcitation 400 nm and λemission 505 nm using a FL600 Microplate Fluorescence Reader. The caspase-3 inhibitor, acetyl-Asp-Glu-Val-Asp-aldehyde was used to exclude nonspecific background in the enzymatic reaction.

Western Blot Analysis. Western blots were performed using a modified procedure (Liu, Y. Y.,et al., (1999) J. Biol. Chem. 274, 1140-1146, Liu, Y. Y.et al., (1999) Exp. Cell Res. 252, 464-470, Watanabe R., et al., (1998) J. Biol. Chem. 273, 9651-9655). Confluent cell monolayers were washed twice with PBS containing 1.0 mM PMSF, and detached with trypsin-EDTA solution. Cells, pelleted by centrifugation, were solubilized in 1.0 ml cold TNT buffer (20 mM Tris-HCl, pH 7.4, 200 mM NaCl, 1.0% Triton X-100, 1.0 mM PMSF, 1.0% aprotinin) for 60 min with shaking. The insoluble debris was excluded by centrifugation at 12,000×g for 45 min at 4° C. The detergent soluble fraction was loaded in equal aliquots, by protein, and resolved using 4-20% gradient SDS-PAGE. The transferred blot was blocked (3% fat-free milk powder in 10 mM Tris-HCl, pH 8.0, 150 mM NaCl, 0.05% Tween-20), and was immuno-blotted with GCS antiserum (1:1000) in binding solution (0.5% BSA in 10 mM Tris-HCl, pH 8.0, 150 mM NaCl) at 4° C. for 18 hr. To detect Xpress tag, P-glycoprotein, and Bcl-2, the antibodies of anti-Xpress tag (1:500), C219 (5 μg/ml) and Ab-1 (2.5 μg/ml), respectively, were used in place of GCS antiserum. Detection employing enzyme-linked chemiluminescence was performed using ECL (Amersham).

Statistics. All data represent the mean±SD. Experiments were repeated two or three times. Student's t-test was used to compare mean values.

### 7.2 Example 1

### Expression of GCS Antisense

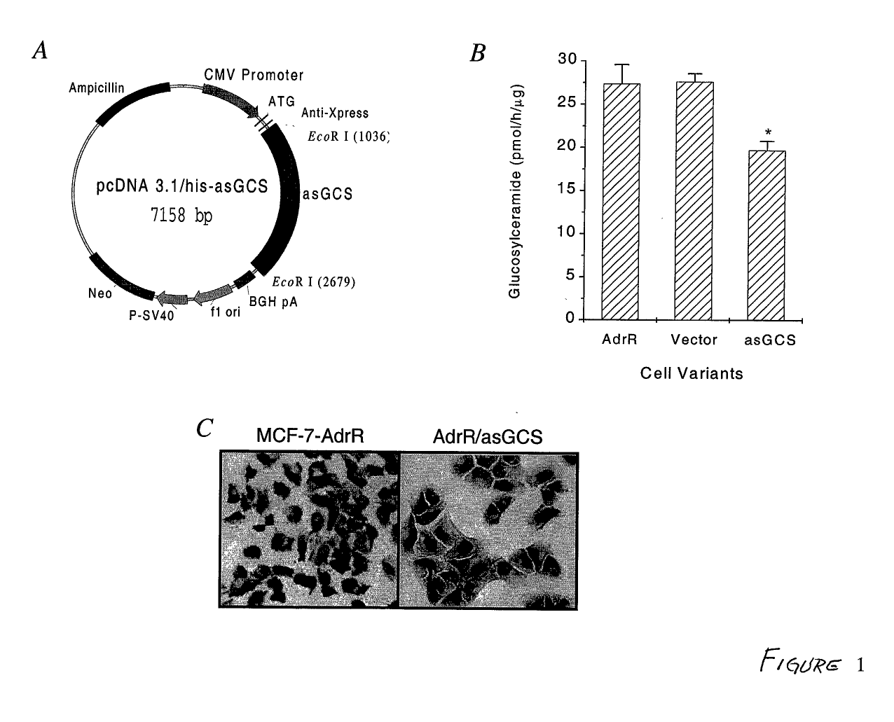

The structure of pcDNA 3.1/his A-asGCS is shown in FIG. 1A. The GCS antisense was cloned into the EcoR I site, just down stream from the anti-Xpress tag sequence in pcDNA 3.1/his A. This plasmid was introduced into MCF-7-AdrR cells by calcium phosphate coprecipitation. G418 was used to select transfectants. It was found that the number of G418-resistant clones in MCF-7-AdrR asGCS transfected cells was much lower than in MCF-7-AdrR cells transfected with pcDNA3.1/his A vector (54/106 vs. 251/106). G418-resistant clones were further selected by measuring GCS activity using the cell-free radioenzymatic assay. In all, fifty-four G418-resistant clones of MCF-7-AdrR asGCS-transfected cells were obtained, and we identified one clone that exhibited a stable 30% decrease in GCS activity (FIG. 1B). Compared with 27.4±2.3 pmol GC synthesized by MCF-7-AdrR parental cells, GCS activity in MCF-7-AdrR/asGCS was decreased to 19.7±1.1 pmol GC (FIG. 1B, p<0.001). There were no differences in GCS activities between the pcDNA 3.1/his A vector-transfected cells and parental MCF-7-AdrR cells (FIG. 1B).

The asGCS-transfected and parental MCF-7-AdrR cells were stained with Giemsa. Representative photomicrographs are shown in FIG. 1C. MCF-7-AdrR/asGCS cells, including nuclei, are flatter and larger than the dome-shaped, more stellate MCF-7-AdrR cells. The asGCS cell line is also more cuboidal with less dense cytoplasm. The population doubling times for both cell lines were similar, 32 and 30 hr for MCF-7-AdrR/asGCS and MCF-7-AdrR cells, respectively.

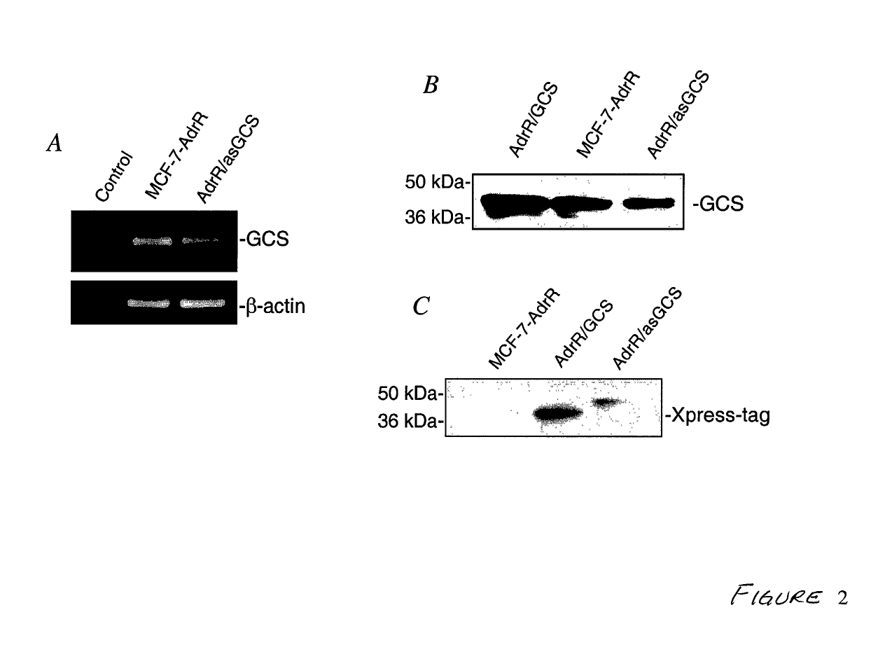

Consistent with diminished GCS activity, GCS mRNA and GCS protein were reduced in MCF-7-AdrR/asGCS cells, compared to MCF-7-AdrR cells. Total mRNA was isolated from both cell lines, and reverse transcribed and amplified through RT-PCR. A representative RT-PCR gel electropherograph is shown in FIG. 2A. As that revealed by densitometric scanning, the mRNA in MCF-7-AdrR/asGCS cells was reduced 3-fold compared to that in MCF-7-AdrR cells, (25.4% vs. 77.5% of β-actin). GCS protein in cell lysates was resolved by SDS-PAGE and identified using GCS antiserum. Western blotting showed that the total amount of GCS protein in MCF-7-AdrR/asGCS cells decreased by 32% compared to MCF-7-AdrR parental cells (77,520 and 112,860 OD units, respectively), FIG. 2B right and center. However, MCF-7-AdrR cells that were transfected with pcDNA3.1his A-GCS, expressed greater amounts of GCS (FIG. 2B, left, AdrR/GCS). In order to evaluate the expression of transfected GCS antisense gene, we employed a Xpress antibody to detect the production of Xpress-GCS fused protein (see FIG. 1A). For a positive control on Western blot, MCF-7-AdrR cells were transfected with sense-orientation pcDNA3.1/his A-GCS vector. This cell line displays 80% higher GCS activity than MCF-7-AdrR cells. We did not find the GCS-Xpress tag in either MCF-7-AdrR or MCF-7-AdrR/asGCS cells (FIG. 2C). However, the tag protein was highly expressed in MCF-7-AdrR GCS transfected cells (FIG. 2C, center). In MCF-7-AdrR/asGCS cells, what appears to be the Xpress-asGCS protein (FIG. 2C, faint band) had a higher molecular weight compared to Xpress-GCS protein of MCF-7-AdrR/GCS and was present at only 15% the level of the latter (FIG. 2C, center).

### 7.3 Example 2

### GCS-antisense Transfected Cell Response to Adriamycin

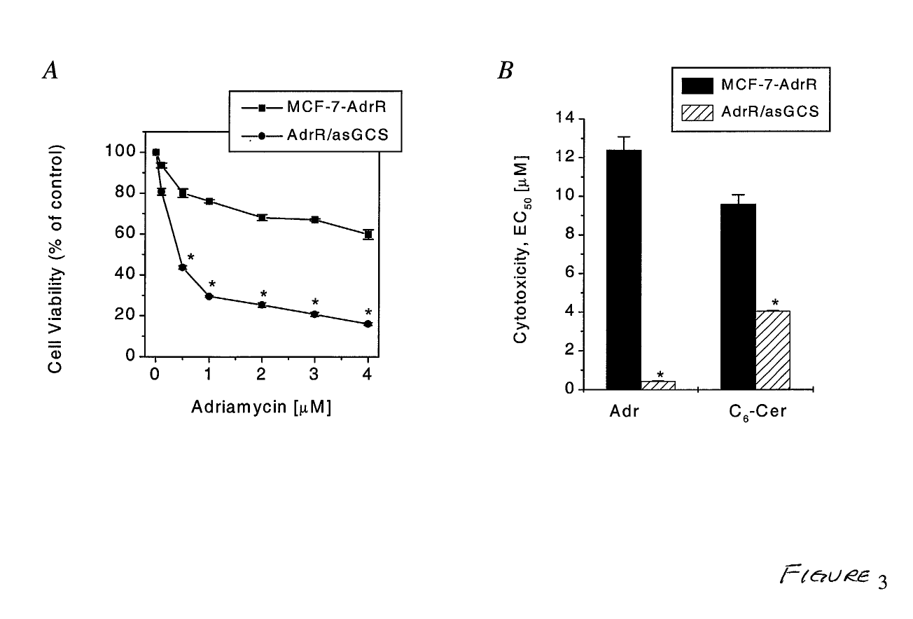

Previous work from our laboratory revealed that overexpression of GCS elicits adriamycin resistance (Liu, Y. Y.et al., (1999) J. Biol. Chem. 274, 1140-1146; U.S. Ser. No. 09/201,115 to Cabot, herein incorporated by reference). After transfection of GCS antisense, adriamycin was used to assess the influence of antisense on cellular response to anthracyclines. Parental and antisense transfected cell lines were treated with increasing concentrations of adriamycin for a three day period. FIG. 3A shows that MCF-7-AdrR/asGCS cells, compared to MCF-7-AdrR cells, were markedly more sensitive to adriamycin. At concentrations of 0.5 μM and higher, survival of MCF-7-AdrR/asGCS cells was significantly lower than MCF-7-AdrR cells (p<0.0001, FIG. 3A). The amount of drug provoking 50% cell death (EC50) was determined. The EC50 of adriamycin decreased 28-fold in MCF-7-AdrR/asGCS cells (0.44±0.01 vs. 12.4±0.7 μM, p<0.0001, FIG. 3B). As expected, we observed that MCF-7-AdrR/asGCS cells were also sensitive to ceramide. At higher concentrations of C6-ceramide (5-10 μM), MCF-7-AdrR/asGCS cell survival was significantly lower than MCF-7-AdrR cells (p<0.0001). The EC50 of C6-ceramide in MCF-7-AdrR/asGCS cells was 2.4-fold less than that observed in MCF-7 AdrR cells (4.0±0.03 vs. 9.6±0.5 μM, p<0.0005, FIG. 3B).

### 7.4 Example 3

### GCS-antisense Transfected Cell Response to Adriamycin

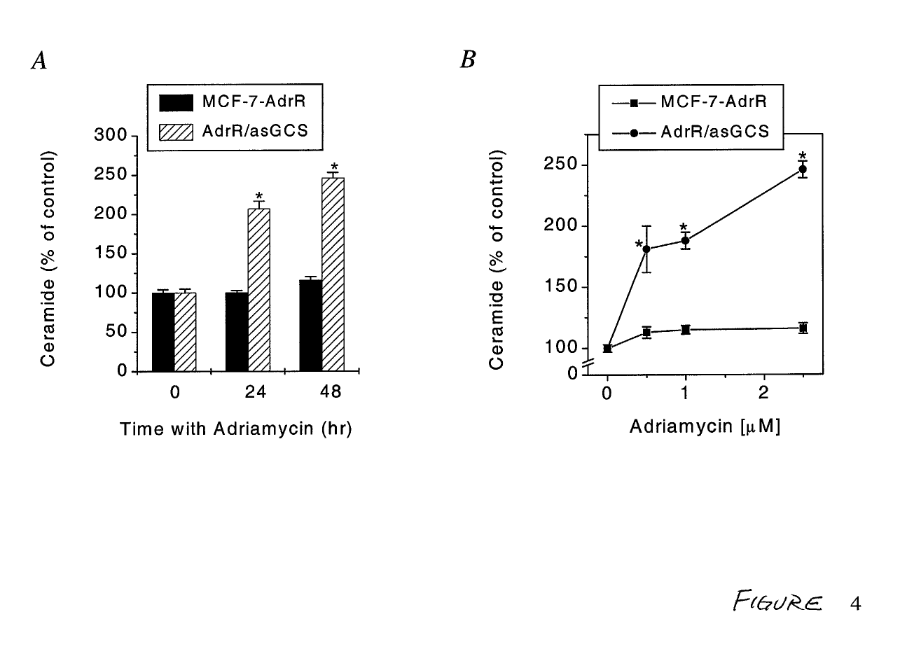

To further elucidate the dynamics of ceramide metabolism in drug sensitivity, ceramide generation was measured in the two cell lines. Adriamycin exposure dramatically elevated ceramide levels in GCS antisense-transfected cells. As shown in FIG. 4, adriamycin treatment increased the levels of ceramide in MCF-7-AdrR/asGCS cells in a time- and dose-dependent manner. At 24 and 48 hr post-treatment, ceramide levels in MCF-7-AdrR/asGCS cells increased 200 and 250%, respectively (FIG. 4A). In sharp contrast, adriamycin treatment did not greatly modify ceramide levels in MCF-7-AdrR cells, which at 48 hr increased only 16% above control. The result of increasing adriamycin dose on ceramide metabolism in the cell lines is shown in FIG. 4B. Adriamycin at 0.5, 1.0, and 2.5 μM enhanced ceramide levels by 181, 188 and 246%, respectively, in MCF-7-AdrR/asGCS cells (FIG. 1B), whereas MCF-7-AdrR cells displayed minimal response over the same dose range.

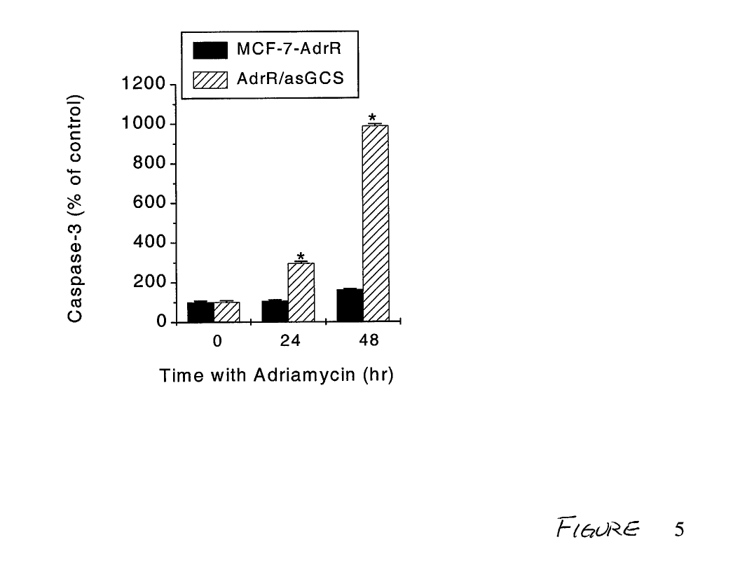

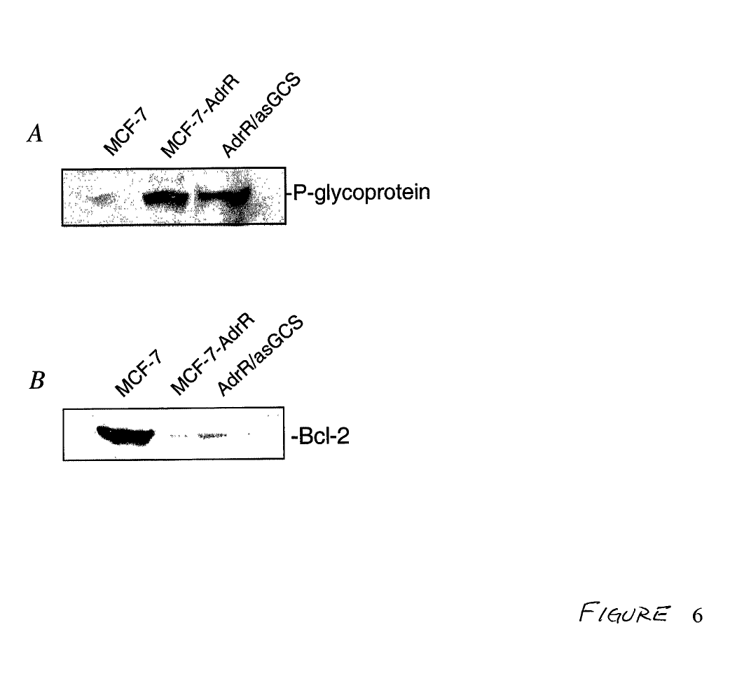

In mammalian cells, ceramide induces apoptosis directly through effector caspases, such as caspase-3 (Yoshimura, S.et al., (1998) J. Biol. Chem. 273, 6921-7, Monney, L., et al., (1998) Eur. J. Biochem. 251, 295-303). To identify whether an alteration in ceramide metabolism in asGCS cells is related to adriamycin sensitivity via signal cascades, we analyzed caspase-3 activity in the parental and transfected cell lines. The data demonstrate that increased effector caspase-3 activity is consistent with changes in ceramide metabolism. At 10 μM doxorubicin, the EC50 in MCF-7-AdrR cells, caspase-3 activity in MCF-7-AdrR/asGCS increased 290 and 980% over control, at 24 and 48 hr, respectively (FIG. 5). In contrast, adriamycin treatment increased caspase-3 by 160% in MCF-7-AdrR cells, albeit only at 48 hr (FIG. 5). In summary, caspase-3 activity in the GCS antisense-transfected cells was 3- and 6-fold greater in response to adriamycin treatment than observed in parental cells (p<0.0001). This suggests that impaired GCS activity permits cells to maintain high levels of ceramide under doxorubicin stress, activating caspase-3 for progression of programmed cell death. Because GCS antisense transfection resulted in enhanced drug sensitivity, we evaluated the expression of P-glycoprotein and Bcl-2. A representative Western blot of P-glycoprotein is shown in FIG. 6A. P-glycoprotein was found only in trace amounts in MCF-7 cells (adriamycin sensitive). Decreased expression of P-glycoprotein was not evident in MCF-7-AdrR/asGCS cells, when compared to the parent MCF-7-AdrR cell line (FIG. 6A). Bcl-2 was found only in trance amounts in MCF-7-AdrR and in MCF-7-AdrR/asGCS cells (FIG. 6B), although Bcl-2 was highly expressed in MCF-7 cells, consistent with our prior finding (Soule, H. D., et al., (1973) J Natl. Cancer Inst. 51, 1409-14016).

Introduction of GCS antisense DNA into chemotherapy-resistant cancer cells reverses cellular resistance to adriamycin and to C6-ceramide in the resulting MCF-7-AdrR/asGCS cell line. The parent line, MCF-7-AdrR was selected from MCF-7 cells by culturing in the presence of adriamycin (Cowan, K. H., et al., (1986) Proc. Natl. Acad. Sci. USA, 83, 9328-32, Fairchild, C. R. et al., (1987) Cancer Res. 47, 5141-5148). These cells exhibit cross-resistance to a wide range of antineoplastic agents including Vinca alkaloids, anthracyclines, and epipodophyllotoxins (Lavie, Y.et al., (1997) J. Biol. Chem. 272, 1682-1687; Cowan, K. H., et al., (1986) Proc. Natl. Acad. Sci. USA, 83, 9328-32; Fairchild, C. R. et al.,. (1987) Cancer Res. 47, 5141-5148; Batist, G., et al. (1986) J. Biol. Chem. 261, 15544-15549; Fairchild, C. R., et al., (1990) Mol. Pharmacol. 37, 801-809; Mimnaugh, E. G., et al, (1991) Biochem. Pharmacol. 42, 391-402;). The MCF-7 human breast cancer cell line (Soule, H. D., Vazguez, J., Long, A., Albert, S., and Brennan, M. (1973) J Natl. Cancer Inst. 51, 1409-14016), in contrast, is drug-sensitive (Lucci, A., Han, T. Y., Liu, Y. Y., Giuliano, A. E. and Cabot, M. C. (1999) Int. J. Onc. 15, 541-546; Lavie, Y.et al., (1997) J. Biol. Chem. 272, 1682-1687; Cowan, K. H., et al., (1986) Proc. Natl. Acad. Sci. USA, 83, 9328-32; Fairchild, C. R. et al.,. (1987) Cancer Res. 47, 5141-5148; Batist, G., et al. (1986) J. Biol. Chem. 261, 15544-15549; Fairchild, C. R., et al., (1990) Mol. Pharmacol. 37, 801-809; Mimnaugh, E. G., et al, (1991) Biochem. Pharmacol. 42, 391-402; Soule, H. D., et al., (1973) J Natl. Cancer Inst. 51, 1409-14016).

After transfection with pcDNA 3.1/his A-asGCS plasmid, MCF-7-AdrR/asGCS cells expressed lower levels of GCS, at both the mRNA and protein level (FIG. 2). GCS enzymatic activity was also found to be lower in MCF-7-AdrR/asGCS cells (FIG. 1B). Due to markedly decreased expression of Xpress-asGCS tag (Western blot, FIG. 2C), it is likely that binding of asGCS mRNA to native GCS mRNA blocks GCS translation and diminishes GCS protein in the antisense transfected cells. The EC50 for adriamycin was reduced 28-fold (FIG. 3B) whereas in the cell-free enzyme assays, GCS activity was reduced by only 30% in MCF-7-AdrR/asGCS cells (FIG. 1B). Several factors, including the existence of GCS isoforms, substrate specificities, and enzyme compartmentalization, may play a role in GCS effects on adriamycin sensitivity. For example, GCS catalyzes ceramide glycosylation, the first step in the biosynthesis of glycosphingolipids (Varki, A. (1993) Glycobiology 3, 97-130). A recent GCS knockout study showed that embryonic lethality was the consequence of homozygosity, revealing a vital role for GCS during development and differentiation in mice (Yamashita et al., (1999) Proc. Natl. Acad. Sci. USA 96: 9142-9147). In present study, G418 survival of the asGCS-transfected clones was minimal compared to survival of the asGCS-free plasmid transfectants. This implies that GCS antisense blocks ceramide glycosylation that is essential for cell development and only the partially blocked clones are able to survive the selection conditions. Additionally, a recent study shows that isoforms of GCS exist with mRNAs corresponding to 3.6 and 3.9 kb (Ichikawa, S. et al., (1998) Biochem. Mol. Biol. Int. 44:1193-1202). Molecular specificity of ceramide has also been demonstrated, as some species, C16-ceramide for example, are more prevalent in apoptosis signaling (Thomas, R. L. et al., (1999) J. Biol. Chem. 274:30580-30588). In addition, cellular ceramide response to DNA damage has been shown to rely on mitochondrion-dependent caspases (Tepper, A. D. et al., (1999) J. Clin. Invest. 103: 971-978).

Ceramide can be generated by de novo biosynthesis and sphingomyelin degradation via the action of sphingomyelinases (Kolesnick, R. N., Kronke, M. (1998) Annu Rev Physiol, 60: 643-665; Hannun, Y. A., and Obeid, L. M. (1995) Trends Biochem Sci. 20: 73-7, Hannun, Y. A. (1996) Science 274:1855-9). Intracellular levels of ceramide are elevated by a variety of stimuli and/or agents that induce apoptosis, including Fas ligand engagement of CD95, ionizing radiation, ultraviolet radiation, chemotherapeutic drugs and genotoxic chemicals, and several cytokines (Kolesnick, R. N., Kronke, M. (1998) Annu Rev Physiol, 60, 643-665, Hannun, Y. A. (1997) Blood 89, 1845-1853, Chuma, S. J., et al., (1997) Cancer Res. 57, 1270-1275, Bose R.,et al.,. (1995) Cell 82, 405-414, Cai, Z., et al., (1997) J. Biol. Chem. 272, 6918-6926, Santana P.et al., (1996) Cell 86, 189-199, Liu, Y. Y., et al., (1999) J. Biol. Chem. 274, 1140-1146, Liu, Y. Y., et al., (1999) Exp. Cell Res. 252, 464-470, Hannun, Y. A. (1996) Science 274,1855-9, Jaffrezou, J. P.et al., (1996) EMBO J. 15, 2417-24, Haimovitz-Friedman, A., (1994) J. Exp. Med. 186, 1831-1841). Ceramide-induced cellular death is one mechanism of adriamycin-induced toxicity (Liu, Y. Y., et al., (1999) J. Biol. Chem. 274, 1140-1146; Lavie, Y., et al.,. (1996) J. Biol. Chem. 271, 19530-19536, Lucci, A., et al., (1999) Cancer 86, 299-310, Spinedi, A., et al.,(1998) Cell Death Differ. 5, 785-791). Cellular ceramide impacts a variety of signaling molecules and pathways (Hannun, Y. A. (1996) Science 274,1855-9). Of these various effects, ceramide induction of the stress-activated protein kinase cascade, and inhibition of complex III activity in the mitochondrial respiratory chain have been linked to the induction of apoptosis (Garcia-ruiz, C., et al.. (1997) J. Biol. Chem. 272, 11369-11377, Verheij, M., et al.,(1996) Nature 380, 75-79, Jarvis, W. D.et al., (1998) Mol. Pharmacol. 54, 844-856). Capspase-3, one of the effector caspases in the stress-activated protein kinase apoptotic signaling pathway, is activated by cell-permeable ceramide as well as endogenous ceramide generated in response to extracellular stimuli (Liu, Y. Y., et al., (1999) Exp. Cell Res. 252, 464-470, Mizushima, N., et al.,. (1996) FEBS Lett. 395, 267-71, Takeda, Y., et al.,. (1999) J. Biol. Chem. 274, 10654-10660). In present study, adriamycin treatment increased cellular ceramide with activation of caspase-3 in the GCS-antisense transfected cells, but not in parental cells. Therefore, the diminished capacity for glycosylation promotes adriamycin-induced cytotoxicity via ceramide-linked activation of caspase-3.

P-glycoprotein, a well-characterized drug resistance mechanism (Gottesman, M. M., and Pastan I. (1993) Annu. Rev. Biochem. 62, 385-427), is highly expressed in MCF-7-AdrR cells (Ichikawa, S., et al., (1996) Proc. Natl. Acad. Sci. U.S.A. 93, 4638-4643). In previous work on the conversion of cells toward drug resistance, increased expression of P-glycoprotein in MCF-7 cells transfected with GCS sense was not observed (Liu, Y. Y., et al.,. (1999) J. Biol. Chem. 274, 1140-1146). Much in line, in the present study we did not observe decreased expression of P-glycoprotein in chemosensitive MCF-7-AdrR/asGCS cells (FIG. 6). This suggests that the reversal of adriamycin resistance conferred by asGCS is not related to P-glycoprotein. Bcl-2 in dephosphorylated form is a strong anti-apoptosis effector involved in ceramide-induced apoptosis signaling pathways (43-45). Increased Bcl-2 does not modulate GCS in MCF-7 cells (Liu, Y. Y., et al., (1999) J. Biol. Chem. 274, 1140-1146), nor, as demonstrated above, was altered Bcl-2 expression found in GCS antisense-transfected MCF-7-AdrR cells. These data suggest that upregulation and down-regulation of GCS regulates adriamycin sensitivity by a mechanism divorced from Bcl-2, introducing asGCS to modulate GCS activity in adriamycin resistant human breast cancer cells, we successfully decreased native GCS expression and restored cellular sensitivity to adriamycin and to C6-ceramide.

Although the present inventiopn has been described in some detail by way of illustration and example for purposes of clarity of understanding, it will be obvious that certain changes and modifications may be practiced within the scope of appended claims.

What is claimed is:

1. A method for reversing drug resistance in a cancer cell, said method comprising introducing an antisense glucosylceramide synthase compoundinto said cell, wherein said introduction reverses drug resistance in said cell.

2. The method of claim 1 wherein said antisense glucosylceramide synthase compound comprises a nucleic acid sequence.

3. The method of claim 2, wherein said nucleic acid sequence is complementary to all or part of a sense strand for glucosylceramide synthase.

4. The method of claim 3, wherein said nucleic acid sequence is between about 15 to about 25 nucleotides in length.

5. The method of claim 1 wherein said cancer cell is selected from the group consisting of a breast cancer cell, prostate cancer cell, ovarian cancer cell, lymphoma cell, melanoma cell, sarcoma cell, leukemia cell, retinoblastoma cell, hepatoma cell, myeloma cell, glioma cell, mesothelioma cell or carcinoma cell.

6. The method of claim 1, further comprising the step of contacting said cell with at least one other agent.

7. The method of claim 6 wherein said agent is a chemosensitizer or chemotherapeutic agent.

8. A method of inducing apoptosis in a cancer cell, said method comprising introducing an antisense glucosylceramide synthase compound into said cancer cell, wherein said introduction induces apoptosis in said cells.

9. The method of claim 7 wherein said antisense glucosylceramide synthase compound comprises a nucleic acid sequence.

10. The method of claim 9, wherein said nucleic acid sequences are complementary to all or part of a sense strand for glucosylceramide synthase.

11. The method of claim 10, wherein said nucleic acid sequence is between about 15 to about 25 nucleotides in length.

12. The method of claim 8 wherein said cancer cell is selected from the group consisting of a breast cancer cell, prostate cancer cell, ovarian cancer cell, lymphoma cell, melanoma cell, sarcoma cell, leukemia cell, retinoblastoma cell, hepatoma cell, myeloma cell, glioma cell, mesothelioma cell or carcinoma cell.

13. The method of claim 8, further comprising the step of contacting said cell with at least one other agent.

14. The method of claim 13 wherein said agent is a chemosensitizer or chemotherapeutic agent.

15. A formulation for reversing drug resistance in a cancer cell or inducing apoptosis in a cancer cell, comprising an antisense glucosylceramide synthase compound and chemosensitizer or chemotherapeutic agent.

16. The formuation of claim 15 wherein said antisense glucosylceramide synthase compound comprises a nucleic acid sequence.

17. The formulation of claim 16 wherein said nucleic acid sequence is complementary to all or part of a sense strand for glucosylceramide synthase.

18. The formulation of claim 17, wherein said nucleic acid sequence is between about 15 to about 25 nucleotides in length.

19. A kit comprising the formulation of claim 15.

20. The kit of claim 19, wherein said antisense glucosylceramide synthase compound comprising said formulation is a nucleic acid sequence.
